(19) United States
(12) Patent Application Publication
(10) Pub. No.: US 2009/0155840 A1
(43) Pub. Date: Jun. 18, 2009

(54) METHOD AND DEVICE FOR CELL COUNTING

(76) Inventors: David J. Beebe, Monona, WI (US); Nisha A. McConnell, Madison, WI (US); Hongmei Yu, San Mateo, CA (US)

Correspondence Address: WISCONSIN ALUMNI RESEARCH FOUNDATION, C/O BOYLE FREDRICKSON S.C, 840 North Plankinton Avenue, Milwaukee, WI, 53203, US

(21) Appl. No.: 11/958,028

(22) Filed: Dec. 17, 2007

(51) Int. Cl. C12Q 1/06; C12M 1/00

(52) U.S. Cl. 435/39; 435/287.1

(57) ABSTRACT

A microfluidic device and method is provided for determining a cell concentration in a sample. The microfluidic device includes a body having a channel therethough that extends along an axis. The channel includes an input and an output, and is at least partially defined by a surface. Indicia overlaps the surface. The channel has a predetermined volume. A portion of the sample is provided in the channel and the cells in the predetermined portions of the channel defined by the indicia are counted.

### FIELD OF THE INVENTION

This invention relates generally to the counting of cells, and in particular, to a method and a device for counting cells within a small sample volume of fluid.

### BACKGROUND AND SUMMARY OF THE INVENTION

Determination of cell concentrations of biological samples is critical for virtually all biological experiments. In most laboratories, cell concentration is determined by using a device such as a hemacytometer or Coulter counter. While these prior devices provide a relatively accurate and reliable method for counting cells, a high cell concentration is required in each sample in order to determine the concentration. For example, a minimum concentration of 10,000 cells per micro-liter is required to make an accurate measurement using a hemacytometer. Consequently, samples with small total cell numbers are often concentrated in very small volumes to achieve an effective cell concentration for measurement. However, even in such circumstances, a significant fraction of the total cells is required to simply perform the measurement, reducing the number of cells left for experimental use. It can appreciated that for samples having very small cell numbers, the use of currently available devices is impractical and restricts the type of analyses that can be done.

By way of example, in most, if not all, assays of somatic stem cell activity, rare cell populations are isolated from their respective tissues and then transplanted or cultured in limiting cell dilutions. The ability of these stem/progenitor cell-enriched populations to produce outgrowths at very low cell numbers is used to estimate stem cell frequency. It is, therefore, critical that the initial cell numbers are estimated correctly and precisely prior to these assays to prevent erroneous results and possible misinterpretation. If the concentrated volume is on the order of 10 micro-liters, the volume required of a hemacytometer, little of the original sample is left after measurement for experimental procedures.

While others have used microfluidic-based devices for cell enumeration and sorting, many of these devices still require the use of relatively large cell numbers/concentrations for accurate detection. Thus, these devices are not acceptable tools for quantifying rare populations of cells, such as stem cells. Moreover, since many of these devices focus specifically on sorting cells based on size or antibody binding, the devices are relatively complex and may require the use of electrically charged fields, infrared lasers, and/or optical tweezers. In addition, while some microfluidic devices which utilize antibody binding to sort specific and rare cell populations could potentially be utilized to analyze stem cell populations, these devices require large initial numbers of cells. Further, these prior devices were designed specifically to be used as an experimental endpoint, which would prevent further use of the sorted rare cell fraction in various stem cell-based assays.

Therefore, it is a primary object and feature of the present invention to provide a method and a device for counting cells within a small sample volume of fluid.

It is a further object and feature of the present invention to provide a method and a device for counting cells that is simple to utilize and inexpensive.

It is a still further object and feature of the present invention to provide a method and a device that allows a user to accurately and reliably count cells in a sample volume of fluid.

In accordance with the present invention, a microfluidic device is provided for determining a cell concentration in a sample. The microfluidic device includes a body having a channel therethough extending along an axis. The channel includes an input and an output and is at least partially defined by a surface. Indicia overlap the surface and the channel has a predetermined volume.

The indicia may define a grid on the surface of the body. Alternatively, the channel is partially defined by first and second spaced sidewalls interconnected by the surface wherein the indicia are lines extending between the first and second walls. The surface may include a plurality of recessed portions axially spaced within the channel. Each recessed portion of the surface is defined by an input end and an output end. The indicia are defined by the input and output ends of the recessed portions of the surface. The predetermined volume of the channel is less than 5 microliters.

In accordance with a further aspect of the present invention, a method of determining a cell concentration in a sample is provided. The method includes the step of providing a channel in a microfluidic device. The channel has an input, an output and a predetermined volume. The channel is filled with the sample and the cells in the channel are counted. Thereafter, the cell concentration is calculated.

The predetermined volume of the channel is less than 5 microliters and the method may include the additional step of providing indicia within the channel. The indicia defines predetermined portions of the channel. The step of counting the cells includes the additional step of determining the number of cells in each of the predetermined portions of the channel. The channel may be partially defined by a surface wherein the indicia are defined by plurality of recessed portions in the surface. Alternatively, the indicia may be a grid.

In accordance with a still further aspect of the present invention, a method is provided of determining a cell concentration in a sample utilizing a microfluidic device having an input and an output. The method includes the steps of filling the channel with the sample and providing indicia for defining predetermined portions of the channel. The cells in the predetermined portions of the channel are counted.

The sample that fills that channel has a predetermined volume, e.g., 5 microliters. The method may include the additional step of calculating the cell concentration. The indicia may be provided within the channel. For example, the channel may partially defined by a surface wherein the indicia are defined by a plurality of recessed portions in the surface. Alternatively, the channel may be partially defined by a surface wherein the indicia is a grid formed in the surface.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an isometric view of an exemplary device in accordance with the present invention;

FIG. 2 is a cross-sectional view of the device taken along line 2-2 of FIG. 1;

FIG. 3 is a cross-sectional view of the device taken along line 3-3 of FIG. 2;

FIG. 4 is an enlarged top plan view showing a portion of the device of FIG. 1;

FIG. 5 an isometric view of an alternate embodiment of a device in accordance with the present invention;

FIG. 6 is an isometric view of a farther alternate embodiment of a device in accordance with the present invention;

FIG. 7 is an isometric view of a still further alternate embodiment of a device in accordance with the present invention;

FIG. 8 is a cross-sectional view of the device taken along line 8-8 of FIG. 7; and

FIG. 9 is a cross-sectional view of the device taken along line 9-9 of FIG. 8.

### DETAILED DESCRIPTION OF THE DRAWINGS

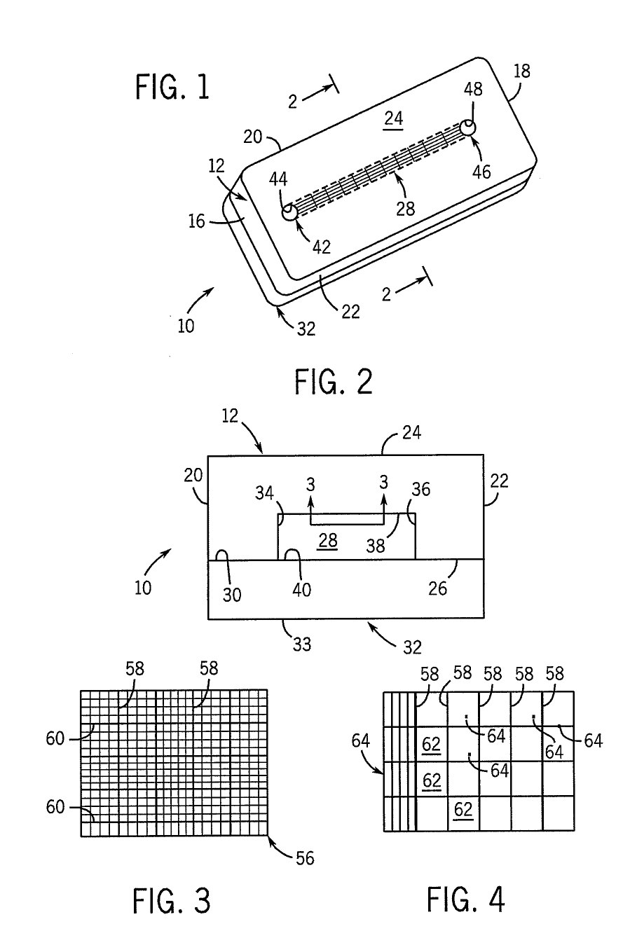

Referring to FIGS. 1-4, a microfluidic device in accordance with the present invention and for effectuating the methodology of the present invention is generally designated by the reference numeral 10. Device 10 includes microfluidic cartridge 12 fabricated from any suitable material such as polystyrene or polydimethylsiloxane (PDMS). Cartridge 12 is defined by first and second ends 16 and 18, respectively, and first and second sides 20 and 22, respectively. Cartridge 12 is further defined by a generally flat upper surface 24 and a lower surface 26. It is intended for lower surface 26 to be positioned on upper surface 30 of substrate 32 so as to define chamber 28 therebetween, as hereinafter described.

Channel 28 extends through device 10 along a longitudinal axis and is defined by first and second spaced sidewalls 34 and 36, respectively, and upper and lower walls 38 and 40, FIG. 2. As such, channel 28 has a known volume. Channel 28 further includes first end 42 that communicates with inlet 44 and second end 46 that communicates with outlet 48. Inlet 44 and outlet 48 communicate with upper surface 24 of cartridge 12. It is contemplated for inlet 44 and outlet 48 of channel 24 to have generally funnel-shaped cross sections to allow for robust and easy mating with a micropipette of a robotic micropipetting station. It is further contemplated for the portions of upper surface 24 about inlet 44 and outlet 48 and for the inner surfaces defining inlet 44 and outlet 48, respectively, to be physically or structurally patterned to contain fluid drops therein.

As best seen in FIGS. 3-4, it is contemplated to provide indicia along upper wall 38 of channel 28 so as define predetermined areas of channel 28. By way of example, it is contemplated to etch or mold graph 56 into upper wall 38 of channel 28. It can be appreciated the that if the height of indicia on upper wall 38 is small compared to the total height of channel 28, then one can neglect the effect of the indicia of the volume of channel 28. If, however, the height of the indices is large (e.g. at least 50% of the height of channel 28), then one would have to take that the height of indicia into account when determining the volume of channel 28. Graph 56 is defined by a plurality of longitudinally spaced lines 58 intersected by a plurality of laterally spaced lines 60, generally perpendicular to longitudinally spaced lines 58. Lines 58 and 60 of graph 56 define a plurality of areas 62 within channel 28.

In operation, a medium having a known volume and containing an unknown number of cells 64 of interest is provided. Channel 28 is filled with a portion of the medium. As heretofore described, the portion of the medium in channel 28 has a known volume given the know volume of channel 28. Cells 64 in the portion of the medium within channel 28 are allowed to settle onto lower wall 40 of channel 28. Using a microscope directed towards upper surface 50 of cartridge 12, a user may view graphical lines 58 and 60, and hence predetermined areas 62, as well as, cells 64 within channel 28. As a result, the user may count the number of cells 64 within each of the predetermined areas 62 defined by graphical lines 58 and 60. Graphical lines 58 and 60 are intended to help the user easily and accurately count cells 64. Thereafter, the user may calculate the number of cells per the known volume of the portion of the medium in channel 28. As such, an estimate of the number of cells 64 in entire volume of the medium may be calculated.

It is contemplated to fabricate upper wall 38 of channel 28 without indicia, as heretofore described. As such, a user may count all of cells within the entire channel 28 without regard to the predetermined areas 62 defined by graphical lines 58 and 60. Thereafter, the user may estimate of the number of cells 64 in entire volume of the medium, as heretofore described. Alternatively, indicia may be incorporated into upper surface 24 of cartridge 12 instead of upper wall 38 of channel 28, as heretofore described, such that the indicia overlap and are in axial alignment with channel 28. Using a microscope directed towards upper surface 24 of cartridge 12, a user may view the indicia, as well as, cells 64 within channel 28. As a result, the user may count the number of cells within each of the predetermined areas defined by the indicia.

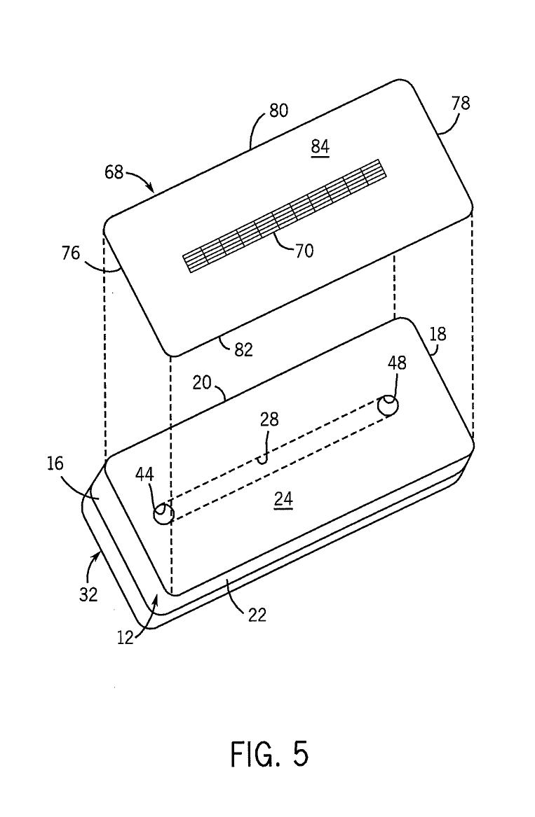

Referring to FIG. 5, sheet 68 having graphical image 70 thereon may be affixed to upper surface 24 of cartridge 12. Sheet 68 is defined by first and second ends 76 and 78, respectively, and first and second sides 80 and 82, respectively. Sheet 68 is farther defined by a generally flat upper surface 84 and a generally flat lower surface. The lower surface of sheet 68 is positioned on upper surface 24 such that first and second ends 76 and 78, respectively, of sheet 68 are aligned with first and second ends 16 and 18, respectively, of cartridge 12 and such that first and second sides 80 and 82, respectively, of sheet 68 are aligned with first and second sides 20 and 22, respectively, of cartridge 12. In addition, it is intended for graphical image 70 to overlap and be in axial alignment with channel 28.

In operation, a medium having a known volume and containing an unknown number of cells 64 of interest is provided. Channel 28 is filled with a portion of the medium. As heretofore described, the portion of the medium in channel 28 has a known volume. Cells 64 in the portion of the medium within channel 28 are allowed to settle on lower wall 40 of channel 28. Using a microscope directed towards upper surface 84 of sheet 68, a user may view the lines of graphical image 70, as well as, cells 64 within channel 28. As a result, the user may count the number of cells within each of the predetermined areas defined by the lines of graphical image 70. Thereafter, the user may calculate the number of cells per the known volume of the portion of the medium in channel 28. As such, an estimate of the number of cells 64 in entire volume of the medium may be calculated.

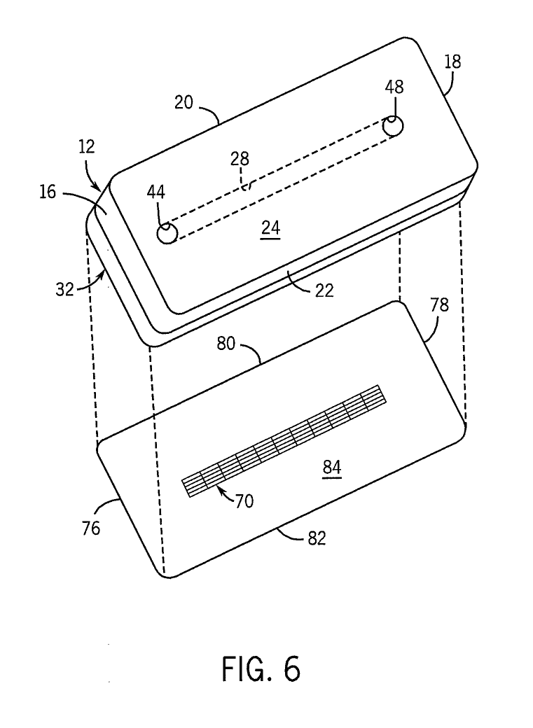

Referring to FIG. 6, sheet 68 having graphical image 70 thereon may be affixed to the lower surface 33 of substrate 32, FIG. 2. Upper surface 84 of sheet 68 is positioned on the lower surface 33 of substrate 32 such that first and second ends 76 and 78, respectively, of sheet 68 are aligned with first and second ends 16 and 18, respectively, of cartridge 12 and such that first and second sides 80 and 82, respectively, of sheet 68 are aligned with first and second sides 20 and 22, respectively of cartridge 12. In addition, it is intended for channel 28 to overlap graphical image 70 and for graphical image 70 to be in axial alignment with channel 28.

In operation, a medium having a known volume and containing an unknown number of cells 64 of interest is provided. Channel 28 is filled with a portion of the medium. As heretofore described, the portion of the medium in channel 28 has a known volume. Cells 64 in the portion of the medium within channel 28 are allowed to settle on lower wall 40 of channel 28. Using a microscope directed towards upper surface 24 of cartridge 12, a user may view the lines of graphical image 70 affixed to the lower surface of substrate 32, as well as, cells 64 within channel 28. As a result, the user may count the number of cells within each of the predetermined areas defined by the lines of graphical image 70. Thereafter, the user may calculate the number of cells per the known volume of the portion of the medium in channel 28. As such, an estimate of the number of cells 64 in entire volume of the medium may be calculated.

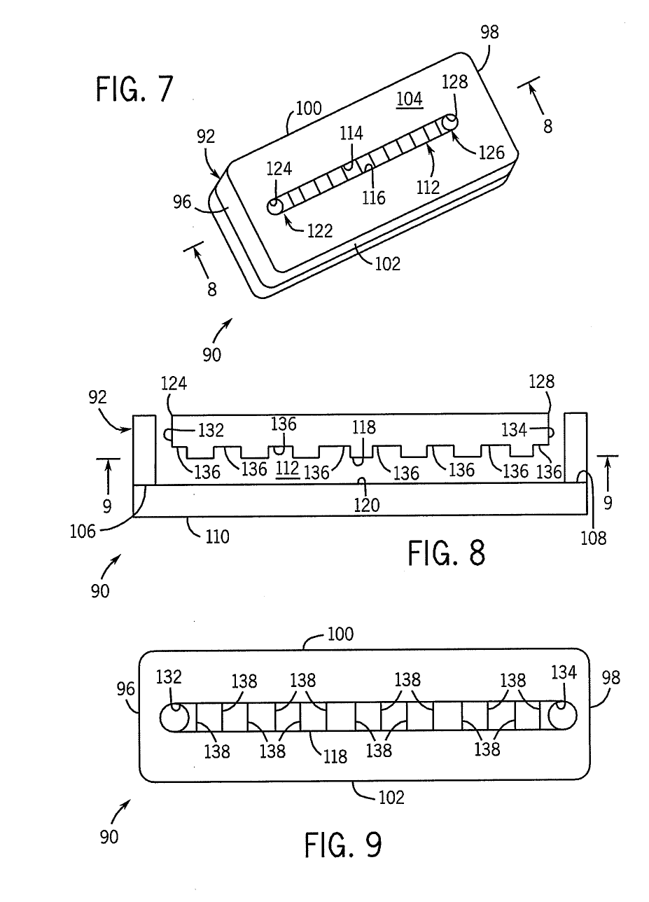

Referring to FIGS. 7-9, an alternate embodiment of a microfluidic device in accordance with the present invention and for effectuating the methodology of the present invention is generally designated by the reference numeral 90. Device 90 includes microfluidic cartridge 92 fabricated from any suitable material such as polydimethylsiloxane (PDMS). Cartridge 92 is defined by first and second ends 96 and 98, respectively, and first and second sides 100 and 102, respectively. Cartridge 92 is further defined by a generally flat upper surface 104 and a lower surface 106. It is intended for lower surface 106 to be positioned on upper surface 108 of substrate 110 so as to define channel 112 therebetween, as hereinafter described.

Channel 112 extends through device 90 along a longitudinal axis and is defined by first and second spaced sidewalls 114 and 116, respectively, FIG. 7, and upper and lower walls 118 and 120, FIG. 8. Channel 112 has a known volume. Channel 112 further includes first end 122 that communicates with inlet 124 and second end 126 that communicates with outlet 128. Inlet 124 and outlet 128 communicate with upper surface 104 of device 90. It is contemplated for inlet 124 and outlet 128 of channel 112 to have generally funnel-shaped cross sections to allow for robust and easy mating with a micropipette of a robotic micropipetting station. It is further contemplated for the portions of upper surface 104 about inlet 124 and outlet 128 and for the inner surfaces 132 and 134 defining inlet 124 and outlet 128, respectively, to be physically or structurally patterned to contain fluid drops therein.

As best seen in FIGS. 8-9, it is contemplated to provide indicia along upper wall 118 of channel 112 so as define predetermined areas of channel 112. By way of example, it is contemplated to provide a plurality of recessed portions 136 in upper wall 118 of channel 112. Recessed portions 136 define by a plurality of longitudinally spaced lines 138 generally perpendicular to the longitudinal axis of channel 112. Adjacent lines 138 on upper wall 118 of channel 112 define indicia for helping a user easily and accurately count cells 64.

In operation, a medium having a known volume and containing an unknown number of cells of interest is provided. Channel 112 is filled with a portion of the medium. As heretofore described, the portion of the medium in channel 112 has a known volume given the known volume of channel 112. The cells in the portion of the medium within channel 112 are allowed to settle on lower wall 120 of channel 112. Using a microscope directed towards upper surface 104 of cartridge 92, a user may view lines 138, as well as, the cells within channel 112. As a result, the user may count the number of cells within each of the areas defined by lines 138. Thereafter, the user may calculate the number of cells per the known volume of the portion of the medium in channel 112. As such, an estimate of the number of cells in entire volume of the medium may be calculated.

Various modes of carrying out the invention are contemplated as being within the scope of the following claims particularly pointing out and distinctly claiming the subject matter, which is regarded as the invention.

We claim:

1. A microfluidic device for determining a cell concentration in a sample, comprising:
a body having a channel therethough along an axis, the channel including an input and an output and being at least partially defined by a surface; and
indicia overlapping the surface;
wherein the channel has a predetermined volume.

2. The microfluidic device of claim 1 wherein the indicia includes a grid on the surface of the body.

3. The microfluidic device of claim 1 wherein the channel is partially defined by first and second spaced sidewalls interconnected by the surface and wherein the indicia are lines extending between the first and second walls.

4. The microfluidic device of claim 1 wherein the surface includes a plurality recessed portions axially spaced within the channel, each recessed portion of the surface defined by an input end and an output end.

5. The microfluidic device of claim 4 wherein the indicia are defined by the input and output ends of the recessed portions of the surface define the indicia.

6. The microfluidic device of claim 1 wherein the predetermined volume of the channel is less than 5 microliters.

7. A method of determining a cell concentration in a sample, comprising the steps of:
providing a channel in a microfluidic device, the channel having an input, an output and a predetermined volume;
filling the channel with the sample;
counting the cells in the channel; and
calculating the cell concentration.

8. The method of claim 7 wherein the predetermined volume of the channel is less than 5 microliters.

9. The method of claim 7 comprising the additional step of providing indicia within the channel, the indicia defining predetermined portions of the channel.

10. The method of claim 9 wherein step of counting the cells includes the determining the number of cells in each of the predetermined portions of the channel.

11. The method of claim 9 wherein the channel is partially defined by a surface and wherein the indicia are defined by plurality of recessed portions in the surface.

12. The method of claim 7 comprising the additional step of providing indicia for defining predetermined portions of the channel.

13. The method of claim 12 wherein the indicia define a grid.

14. A method of determining a cell concentration in a sample utilizing a microfluidic device having an input and an output; comprising the steps of:
filling the channel with the sample;
providing indicia for defining predetermined portions of the channel; and
counting the cells in the predetermined portions of the channel.

15. The method of claim 14 wherein the sample that fills that channel has a predetermined volume.

16. The method of claim 15 comprising the additional step of calculating the cell concentration.

17. The method of claim 15 wherein the predetermined volume of the sample is less than 5 microliters.

18. The method of claim 14 wherein the indicia is provided within the channel.

19. The method of claim 14 wherein the channel is partially defined by a surface and wherein the indicia are defined by plurality of recessed portions in the surface.

20. The method of claim 14 wherein the channel is partially defined by a surface and wherein the indicia is a grid formed in the surface.
